(19) United States
(12) Patent Application Publication
(10) Pub. No.: US 2012/0046666 A1
(43) Pub. Date: Feb. 23, 2012

(54) ANCHORING CATHETER SHEATH

(76) Inventor: George Klein, London (CA)

(21) Appl. No.: 13/202,854

(22) PCT Filed: Feb. 24, 2010
(86) PCT No.: PCT/CA10/00263

§ 371 Date: Nov. 9, 2011

Related U.S. Application Data
(60) Provisional application No. 61/155,046, filed on Feb. 24, 2009.

(51) Int. Cl. A61B 17/00

(52) U.S. Cl. 606/108

(57) ABSTRACT

In general, the invention provides improved sheaths for enhanced control over the relative position of the sheath or inserted catheter relative to a biological tissue. The invention also provides improved sheaths for controlling the longitudinal and axial movement of inserted catheters relative to the sheath. The sheaths include an active anchor at the distal end capable of reversibly adhering the sheath to a tissue. Exemplary active anchors include a reversibly inflatable balloon, a deflectable tip, a suction cup, a screw, and a barb.

### CROSS-REFERENCE TO RELATED APPLICATIONS

This application claims benefit of U.S. Provisional Application No. 61/155,046, filed Feb. 24, 2009, which is hereby incorporated by reference.

### BACKGROUND OF THE INVENTION

The invention relates to the field of medical devices and methods of their use.

Catheter introducers and guiding sheaths are devices that assist in guiding and stabilizing catheters and instruments within the heart and other organs. Sheaths are generally hollow tubes with pre-formed curvature sections that are introduced into biological lumens, e.g., the vascular system, and then guided to an appropriate position under fluoroscopic visualization. A catheter is then inserted and advanced through the distal end of the sheath to the target. One common use for a cardiac sheath is in the procedure to achieve cardiac ablation, where the catheter tip must be directed to a specific point inside the heart and kept in a stable position during the application of ablative (e.g., RF or cryo) energy.

While current cardiac sheaths are acceptable for most applications, they can have a number of disadvantages. They often do not have appropriate curvature to allow catheters to reach difficult parts of the cardiac anatomy and often do not have sufficient stability to keep the catheter tip in optimal contact with the target zone, which in ablation is the endocardial surface of the heart as the heart is beating. These problems are particularly significant during cardiac ablation, especially during ablation within the left atrium.

Current catheter sheaths allow the physician to rotate the catheter about its longitudinal axis following insertion. For catheters with pre-curved distal sections, this allows the curvature to be aimed in the correct direction in order to continue to advance the catheter towards its intended target. In other cases, the catheter may be rotated in order to position the distal tip at the correct target in order to perform ablation or to perform some other diagnostic or therapeutic maneuver. Current catheter sheaths do not, however, restrict catheter rotation following this positioning step (i.e., when the physician sets the sheath aside), and they often rotate inadvertently without the intervention of the physician. This results in the catheter and the sheath curvatures becoming “out of plane” with each other making catheter placement difficult or resulting in the catheter “slipping” off the target. Alternately, the operator may wish the catheter to be out of plane with the sheath, for example the sheath bending in one plane and the catheter bending rotated 90 degrees from that plane. Furthermore, current catheter sheaths do not provide any mechanism to indicate to the physician the relative position of the sheath and catheter curvatures, nor to stabilize it in the desired position.

Even when current catheter sheaths position the catheter correctly, there are often nearby structures that can be damaged by the catheter or by the application of ablative energy through that catheter. For example, a catheter positioned on the epicardial surface of the heart could cause damage to the pericardium or the phrenic nerve. Current catheter sheaths do not assist in keeping or moving these structures away from the catheter.

Accordingly, there is a need for new sheaths.

### SUMMARY OF THE INVENTION

In general, the invention provides improved sheaths for enhanced control over the relative position of the sheath or inserted catheter relative to a biological tissue. The invention also provides improved sheaths for controlling the longitudinal and axial movement of inserted catheters relative to the sheath.

In one aspect, the invention provides an intraluminal sheath having a proximal end, a distal end, and a lumen sized to allow passage of a catheter and extending from the proximal end to the distal end and including an active anchor at the distal end capable of reversibly adhering the sheath to a tissue to provide substantially constant relative position of the sheath to the tissue. Examples of active anchors are a reversibly inflatable balloon, a deflectable tip, a suction cup, a screw, and a barb. The anchor may further include a sensor, e.g., an electrode or pressure or temperature sensor. In preferred embodiments, the sheath is sized for percutaneous access to the interior of a human heart or the sheath is sized for percutaneous access to a human epicardium via an introducer of 10 gauge or smaller diameter.

The sheath may further include a side exit for a catheter at the distal end. The distal end may be fixed curve or variably curved. The sheath may also include fiducial marks to indicate the axial position of a catheter relative to the sheath.

In embodiments in which the anchor is a balloon or suction cup, the sheath includes another lumen for inflating and deflating the balloon or providing and releasing suction. For other anchors, the sheath includes a control to actuate the anchor, e.g., via electrical, mechanical, or pneumatic control. In certain embodiments, the sheath may include two or more anchors, which may operate by the same or different mechanisms. For example, a deflectable tip may further include a screw or barb for fixation to tissue.

Sheaths of the invention may also include a lock to prevent axial and/or longitudinal movement of a catheter relative to the sheath. Exemplary locks include a tab or slot that mates with a corresponding tab or slot on a catheter. Another lock is a clamp capable of applying radial pressure to a catheter. Such a lock may have a high degree of static friction between the sheath and the catheter, e.g., via a detent.

The invention further features a method of positioning a catheter in a lumen of a subject by introducing a sheath of the invention into a subject; activating the active anchor to adhere the sheath to a tissue adjacent the lumen to provide substantially constant relative position of the sheath to the tissue; and inserting a catheter into the sheath so that the catheter traverses the sheath to the distal end.

Exemplary lumens are within a blood vessel or organ, e.g., heart or lung, of the subject. In the methods, the catheter may be any appropriate catheter for the medical use, e.g., an interventional or diagnostic catheter. The catheter may also be employed to deliver a fluid to the lumen or remove a fluid or other tissue from the lumen.

In certain embodiments, the anchor may also displace a second tissue away from the catheter, as described herein.

Exemplary size, lengths, and uses of sheaths of the invention are provided herein.

Other features and advantages will be apparent from the following description, the drawings, and the claims.

By “subject” is meant any animal, e.g., a human, other primate, other mammal, a bird, a reptile, or an amphibian.

By “active anchor” is meant an anchor requiring actuation, e.g., by a physician, to adhere to a tissue.

By “side exit” is meant an opening adjacent to and not coincident with the distal end of a sheath.

By “high degree of static friction” is meant static friction of sufficient magnitude so objects held by it do not move relative to each other without actuation, e.g., application of torque by a physician.

### BRIEF DESCRIPTION OF THE DRAWINGS

FIG. 1 is a photograph of a cardiac sheath.

FIG. 2 is a photograph of a curved distal end of a cardiac sheath.

FIG. 3 is a photograph of an electrophysiology catheter.

FIG. 4 is a photograph of the distal end of an electrophysiology catheter.

FIG. 5 is a photograph of a catheter inserted into a sheath.

FIG. 6 is a photograph of a catheter exiting the distal end of a sheath, with their curvatures in plane.

FIG. 7 is a photograph of a catheter exiting the distal end of a sheath, with their curvatures out of plane.

FIG. 8 is a photograph of the proximal end of a catheter inserted in a sheath.

FIG. 9 is a schematic depiction of a sheath having a balloon anchor.

FIG. 10 is a schematic depiction of a sheath having a deflectable tip anchor.

FIG. 11 is a schematic depiction of a sheath having a suction cup anchor.

FIG. 12 is a schematic depiction of a sheath having a screw anchor.

FIG. 13 is a schematic depiction of a sheath having a barb anchor.

FIG. 14 is a schematic depiction of a sheath having a lock to control axial rotation of the catheter relative to the sheath and a secondary lumen to actuate a balloon or suction cup anchor.

FIG. 15 is a schematic depiction of a sheath having a lock to control axial rotation of the catheter relative to the sheath and an actuator for a deflectable tip or screw anchor.

FIG. 16 is a schematic depiction a catheter and sheath having fiducial marks to indicate their relative alignment.

FIG. 17-1 is a schematic depiction of side and end views of a catheter and sheath with their curvatures in plane.

FIG. 17-2 is a schematic depiction of side and end views of a catheter and sheath with their curvatures out of plane.

FIG. 18-1 is a schematic depiction of lock employing a chuck to prevent axial and longitudinal motion of the catheter relative to the sheath.

FIG. 18-2 is a schematic depiction of lock employing a tab and groove prevent axial but not longitudinal motion of the catheter relative to the sheath.

### DETAILED DESCRIPTION OF THE INVENTION

The present invention provides improved sheaths for insertion of catheters into lumens of subjects, with one or more anchor mechanisms used alone or in combination and allowing the sheath to be anchored into position relative to tissue adjacent to the lumen, e.g., in the heart. The sheaths allow access to difficult cardiac anatomy, and the distal portion of the sheath moves with the tissue to which it is anchored, providing additional stability to the catheter, e.g., during the heart cycle. It specifically allows stabilization and guidance of ablation sites required around an orifice, such as the tissue around the outside of a pulmonary vein (“antral” area). The invention further provides an improved sheath with a locking mechanism to prevent inadvertent catheter movement, i.e., axially or longitudinally, and with an indicator mechanism to document the current axial and/or longitudinal position of the catheter. The sheath may be fixed curve or variably deflectable, and the catheter may be fixed curve or variably deflectable, e.g., as described in U.S. Pat. Nos. 4,601,705, 4,960,134, 6,066,126, and 2005/0267462.

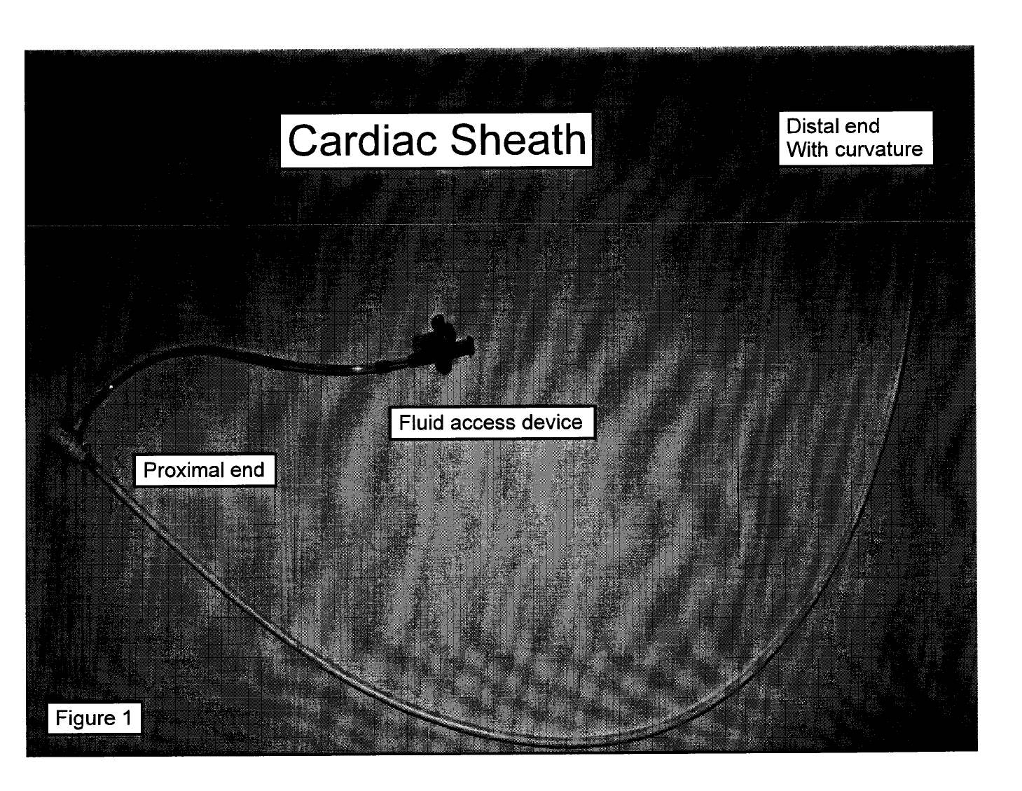

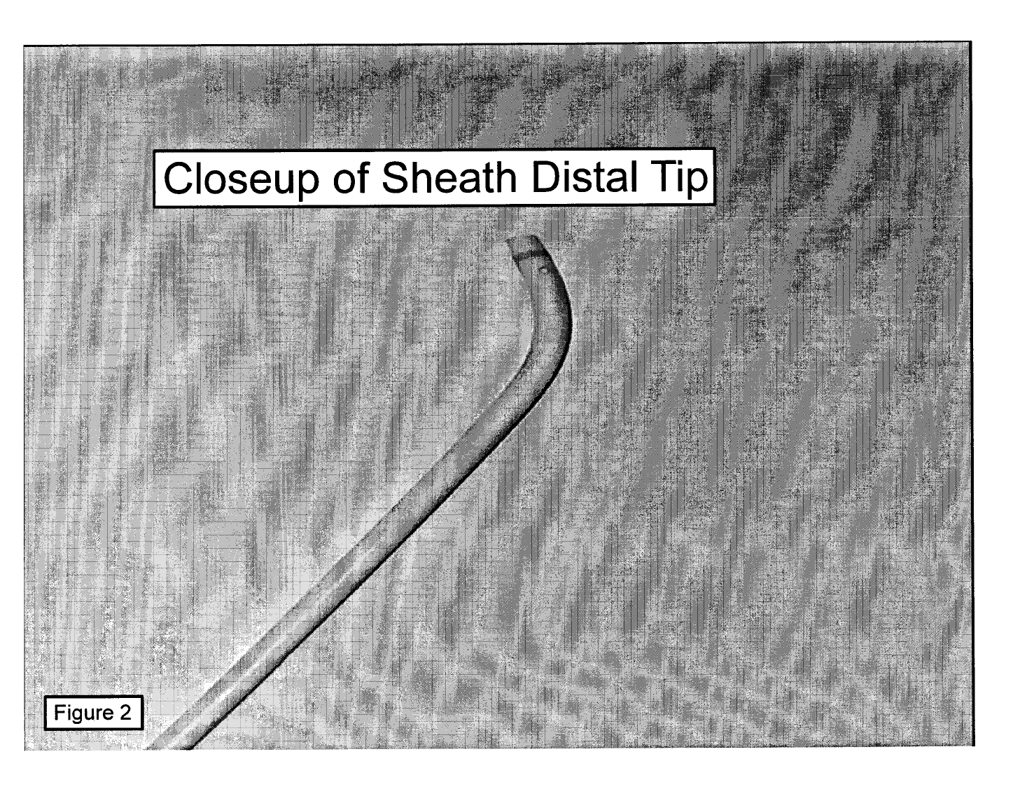

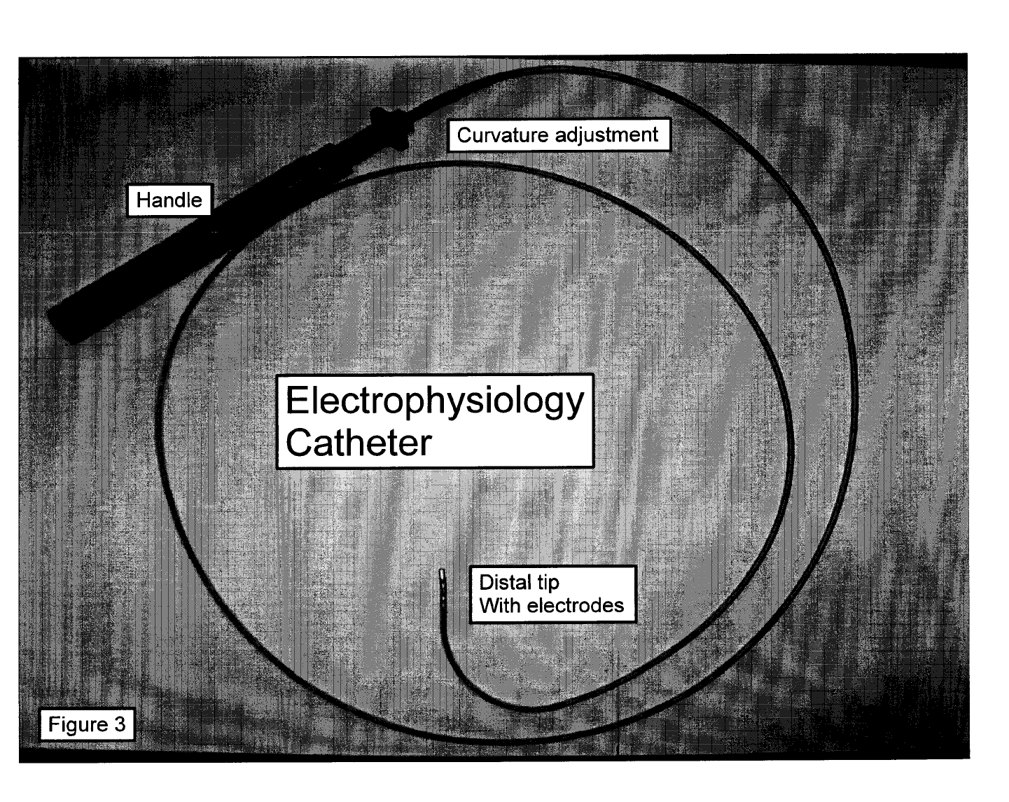

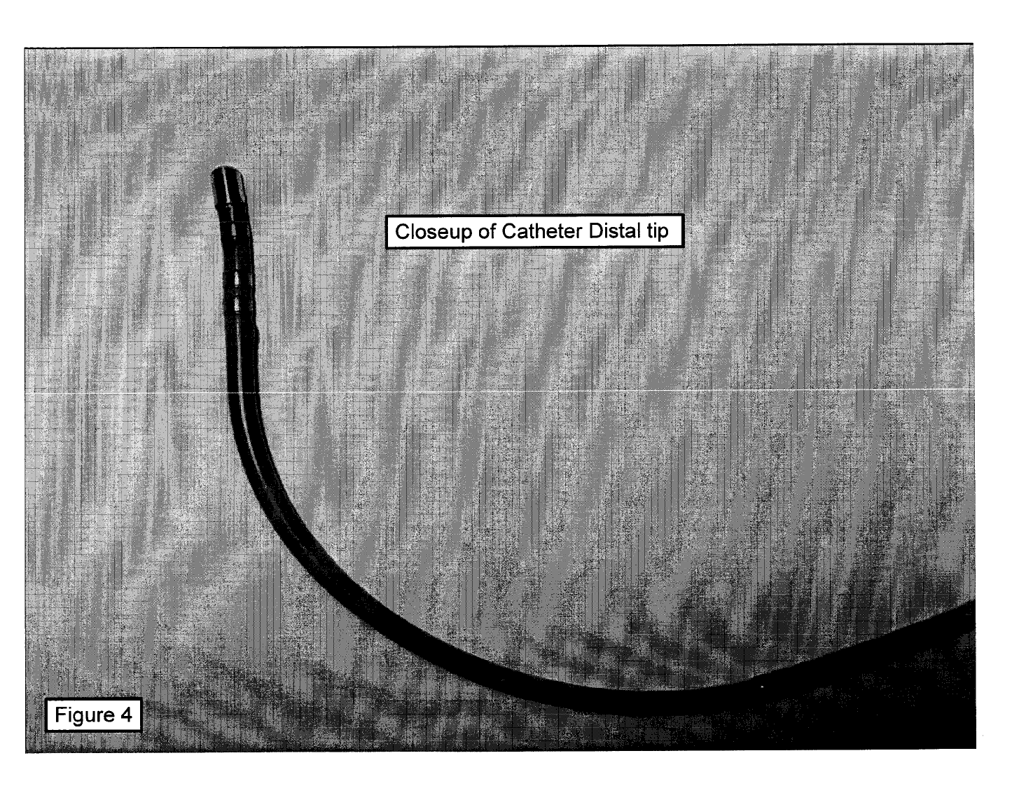

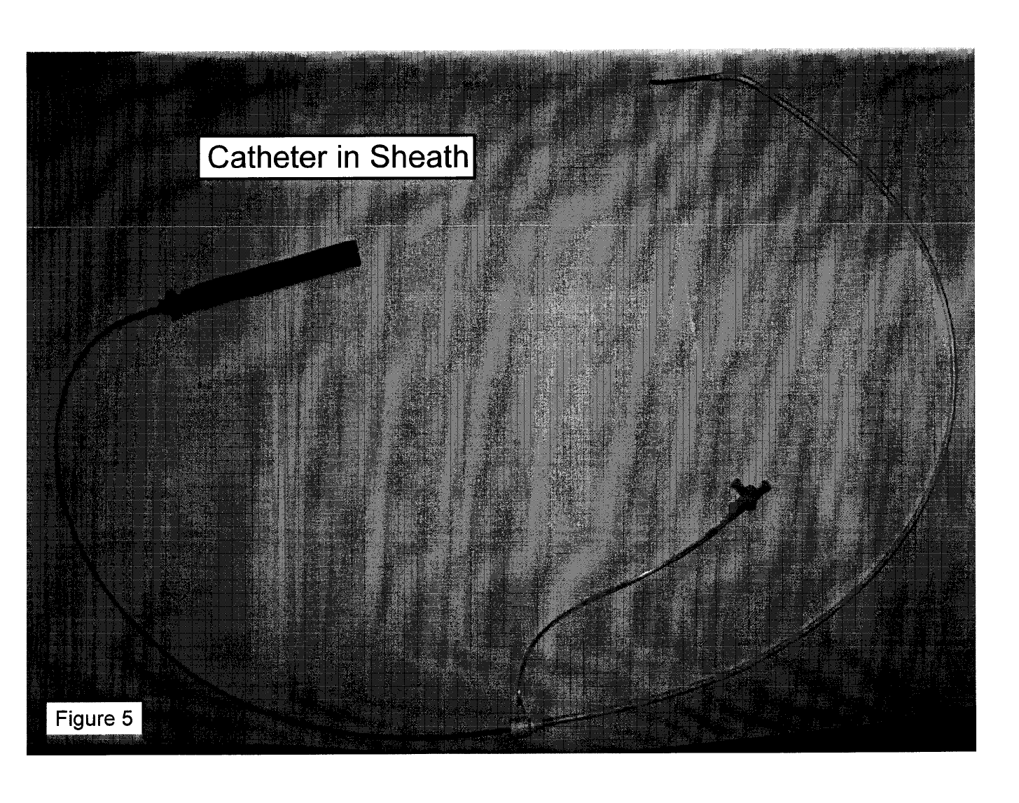

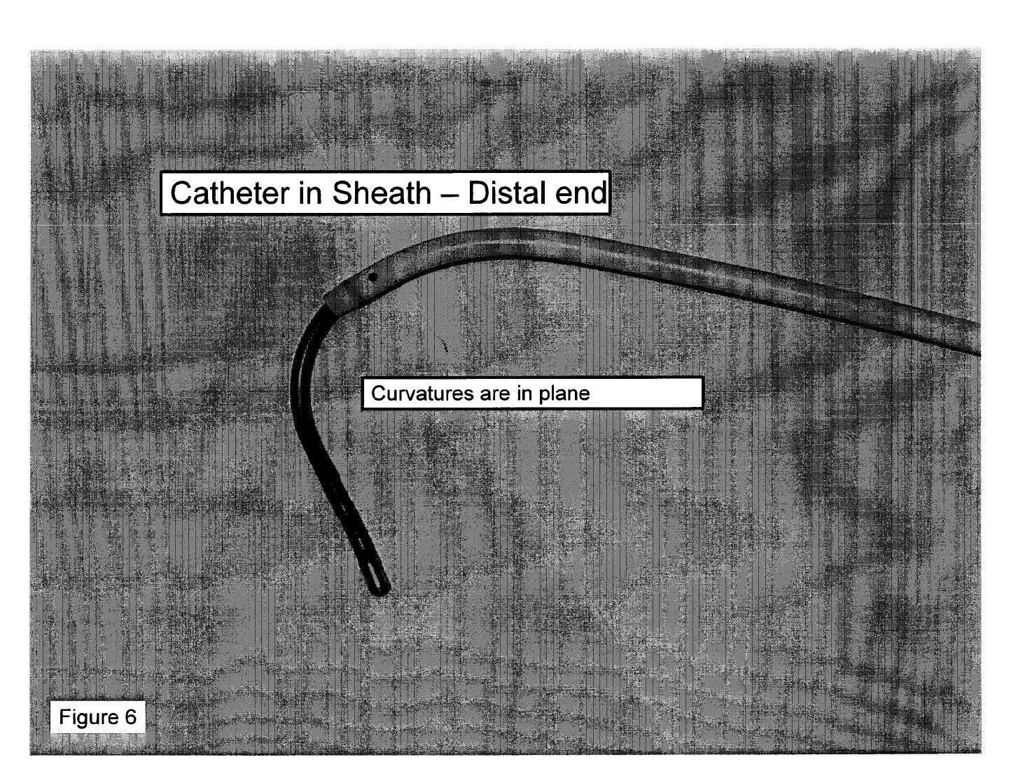

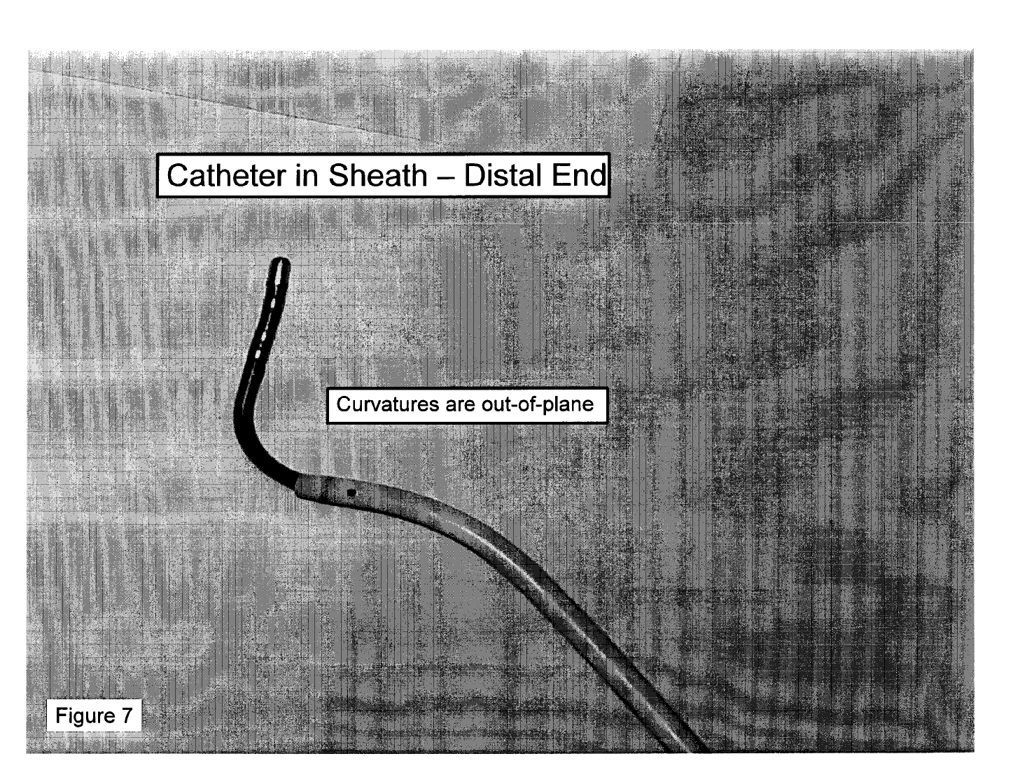

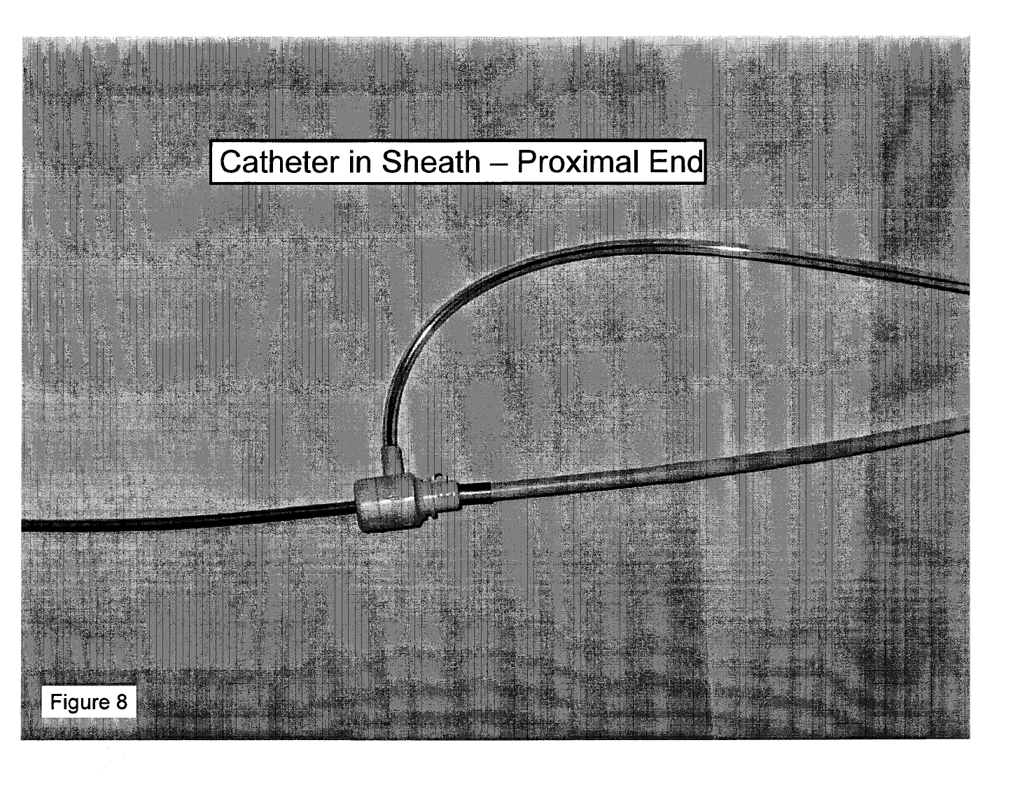

FIGS. 1-8 provide an overview of basic sheath and catheter structure. FIG. 1 shows a typical sheath, without an active anchor, and FIG. 2 shows an example of a fixed curve distal tip of a sheath. FIG. 3 shows a typical electrophysiology catheter, and FIG. 4 shows an example of the distal tip of the catheter. FIG. 5 illustrates how a catheter is inserted into a sheath. FIGS. 6 and 7 show a curved catheter and sheath with the curvatures in plane (6) and out of plane (7). FIG. 8 shows the proximal ends of a catheter inserted into a sheath.

Anchor Mechanism

The invention provides a stable platform for a diagnostic, ablation, or interventional catheter in any intravascular or organ space. The anchors employed are active, i.e., requiring actuation, and thus the sheath also contains the necessary controls for actuation, e.g., electrical, mechanical, or pneumatic.

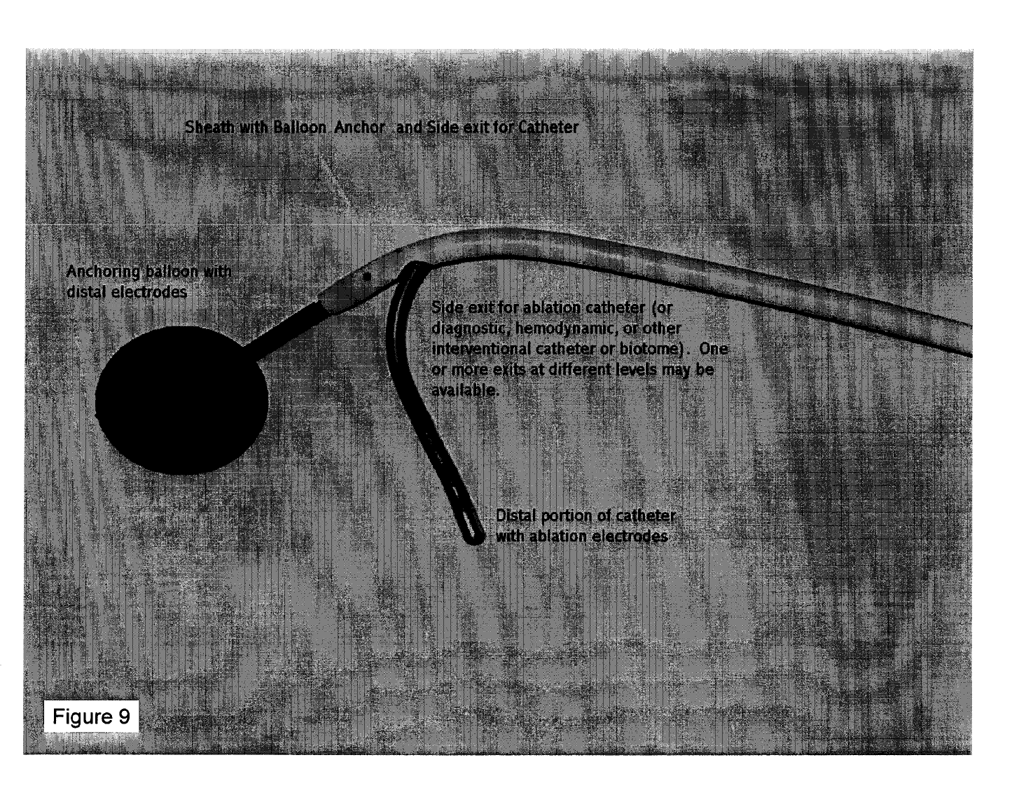

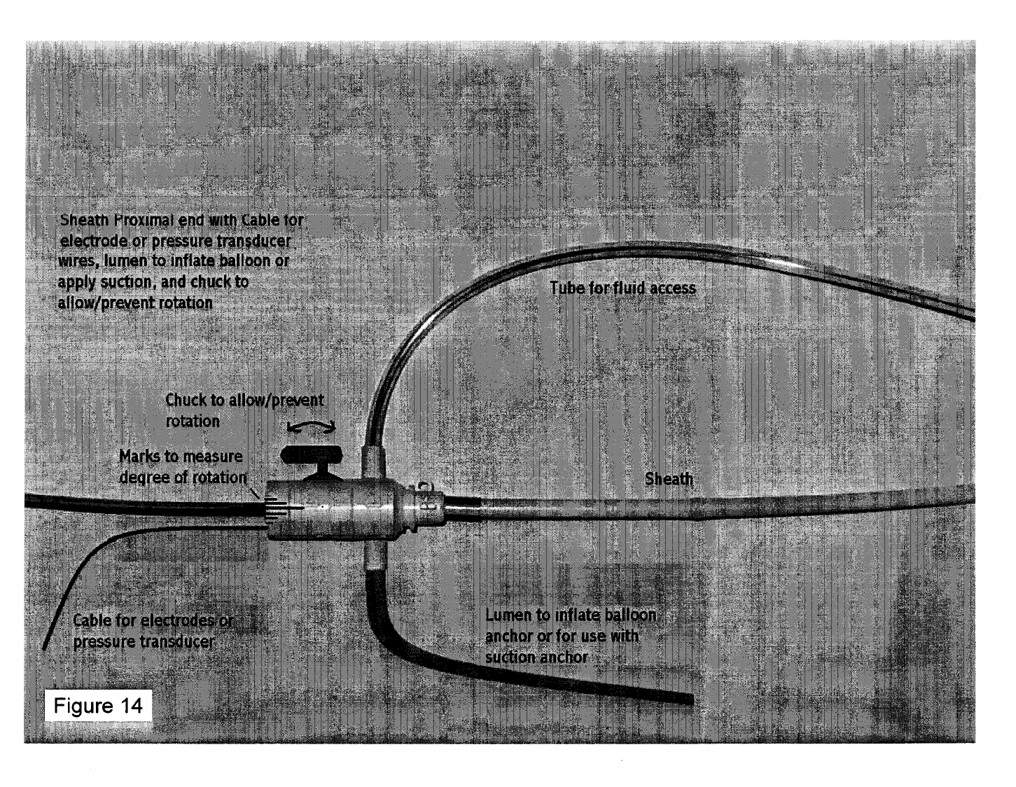

In one embodiment the anchor mechanism is a balloon that can be inflated to lodge the sheath against a tissue (FIG. 9). For example in ablation of atrial fibrillation, the balloon is inflated at or inside the os of a pulmonary vein to allow stability and a constant location to allow an ablation catheter exiting from the sheath proximal to the balloon to create a circular lesion around the antrum of the vein. This balloon could also keep unwanted or interfering anatomy from the proximity of the distal end of the sheath, preventing damage to that anatomy by the catheter or by energy delivered through that catheter. For example, ablation of epicardium in the pericardial space often results in unnecessary and potentially harmful ablation of the pericardium and non cardiac structures such as the phrenic nerve in close association with the pericardium. The balloon allows the ablating electrode to be directed at the epicardium while keeping the pericardium away from the ablating tip. For this embodiment, the sheath includes a lumen for the introduction and removal of fluid from the balloon (FIG. 14). Balloons suitable for this purpose are known in the art, e.g., U.S. Pat. Nos. 5,800,450, 6,314,462, 6,475,226, and 6,491,710.

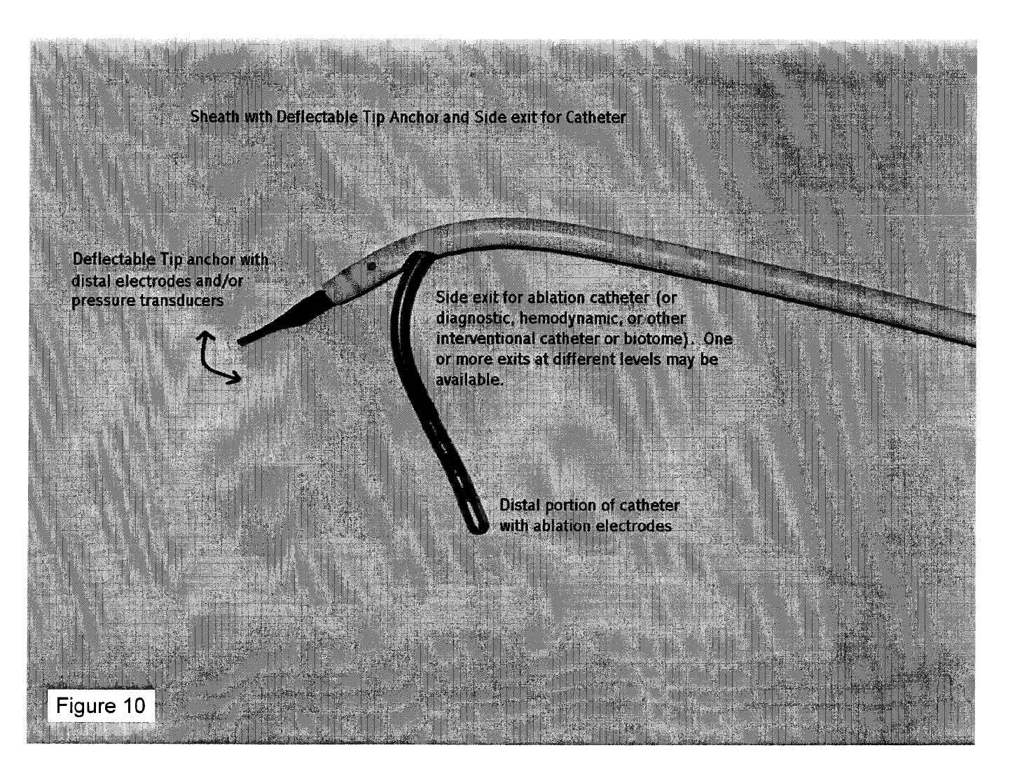

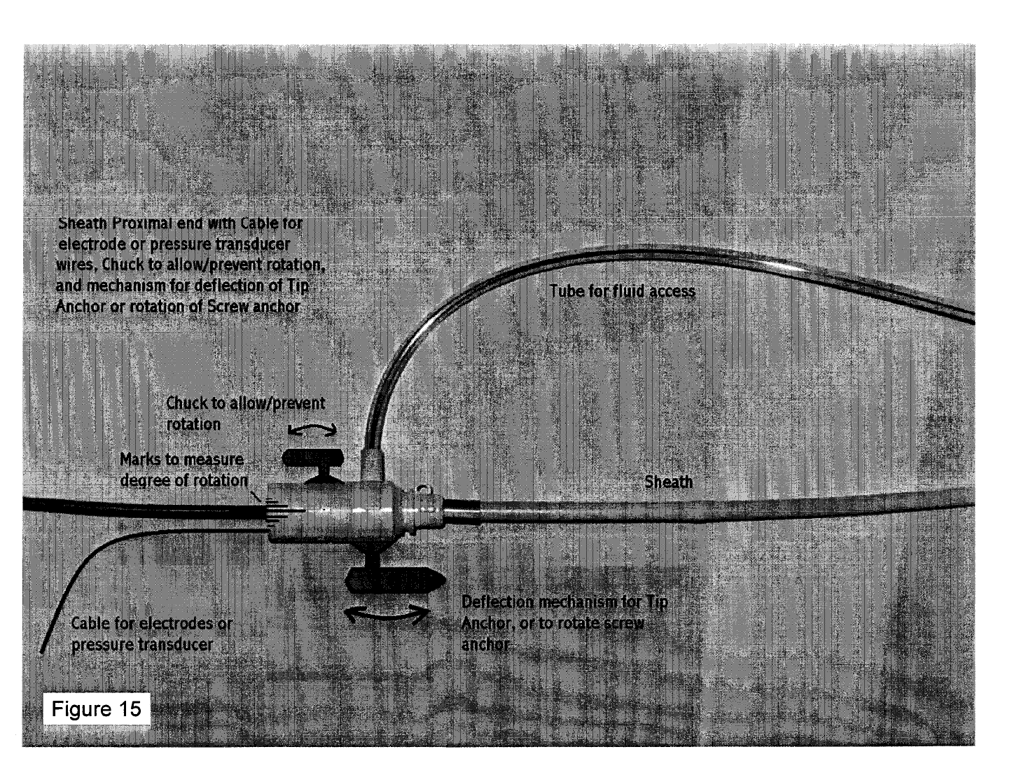

In a second embodiment, the anchor mechanism is a deflectable tip that is positioned against a convenient anatomical structure (FIG. 10). This tip may include additional active fixation mechanisms, e.g., barbs or screws as described herein, that can be embedded in the tissue. For example, in ablation of atrial flutter, where a line of block must be made across the isthmus between the tricuspid valve and the inferior vena cava, the deflectable tip is positioned to hang on a ledge, such as the tricuspid portion of the tricuspid caval isthmus. The sheath further includes a deflection mechanism at the proximal end to allow the user to deflect the tip (FIG. 15).

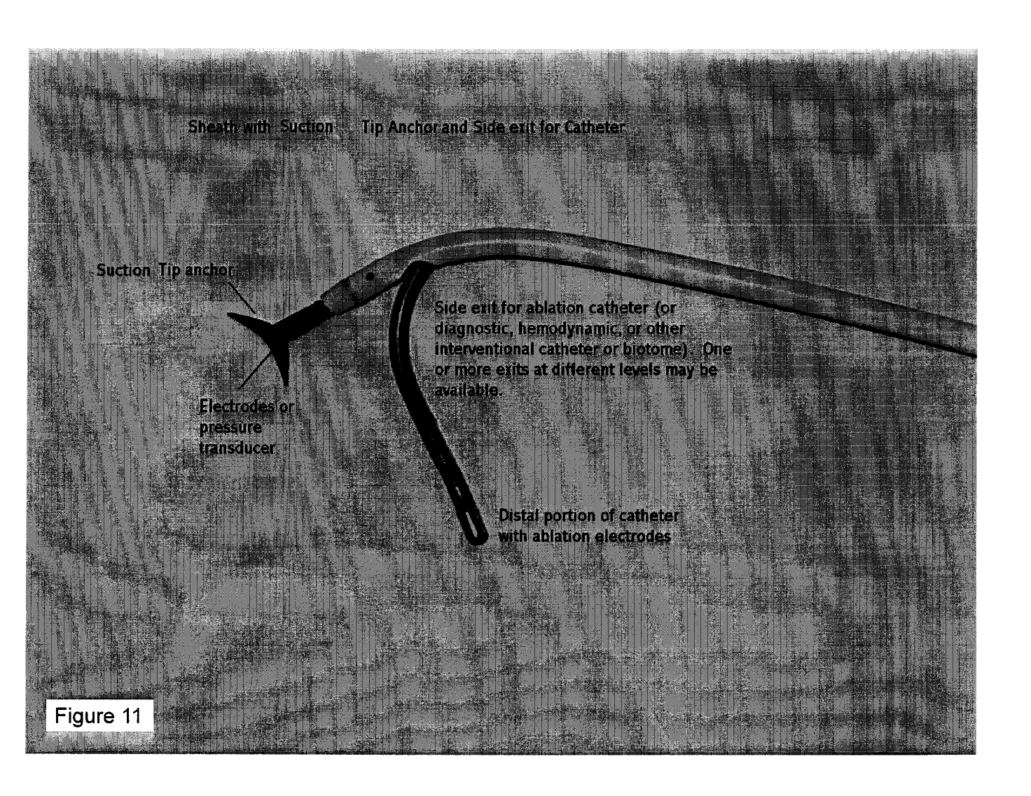

In another embodiment, the mechanism includes a suction cup to attach to a flat tissue, e.g., to allow a stable platform to ablate above or below or circumferentially around it (FIG. 11). As with the balloon anchor, the sheath for a suction cup includes a lumen for introducing or removing fluid to reduce pressure or normalize pressure (FIG. 14). Suitable suction cups are known in the art, e.g., U.S. Pat. Nos. 4,723,940 and 6,314,962.

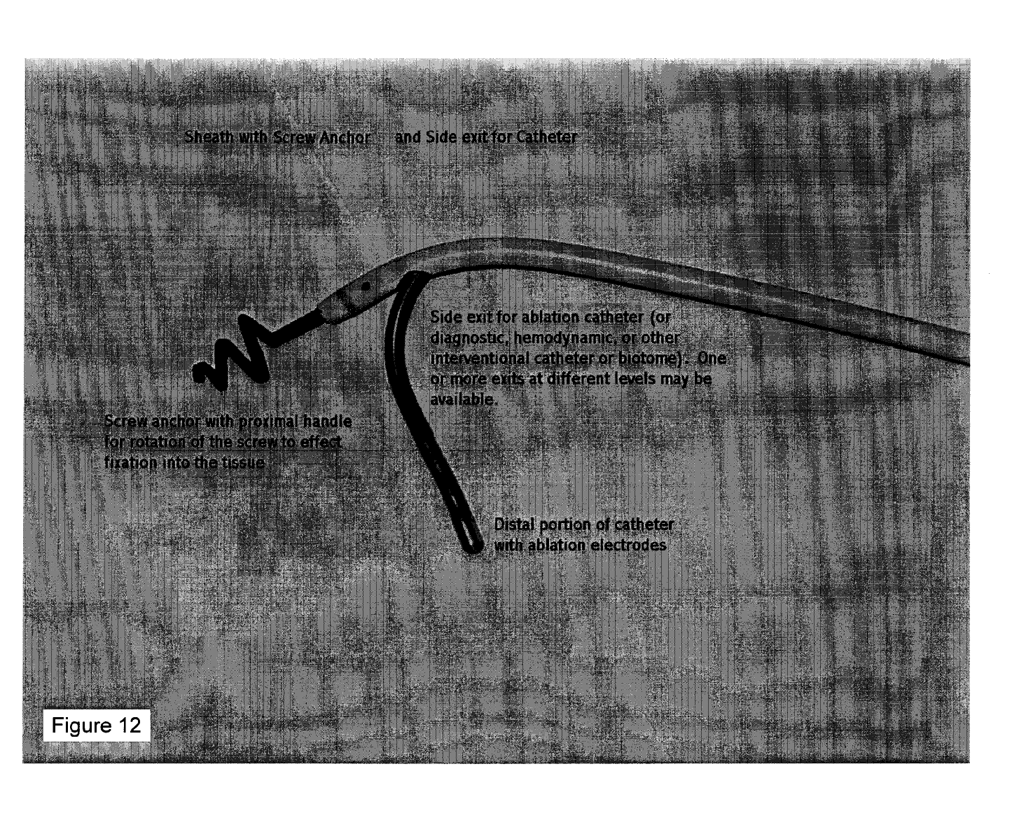

A further embodiment employs a spiral screw that is directly fixated into the tissue (FIG. 12). As with the deflectable tip, the sheath includes an actuator at the proximal end to allow the user to introduce the screw into the tissue (FIG. 15). Suitable screws are known in the art, e.g., U.S. Pat. No. 4,000,745.

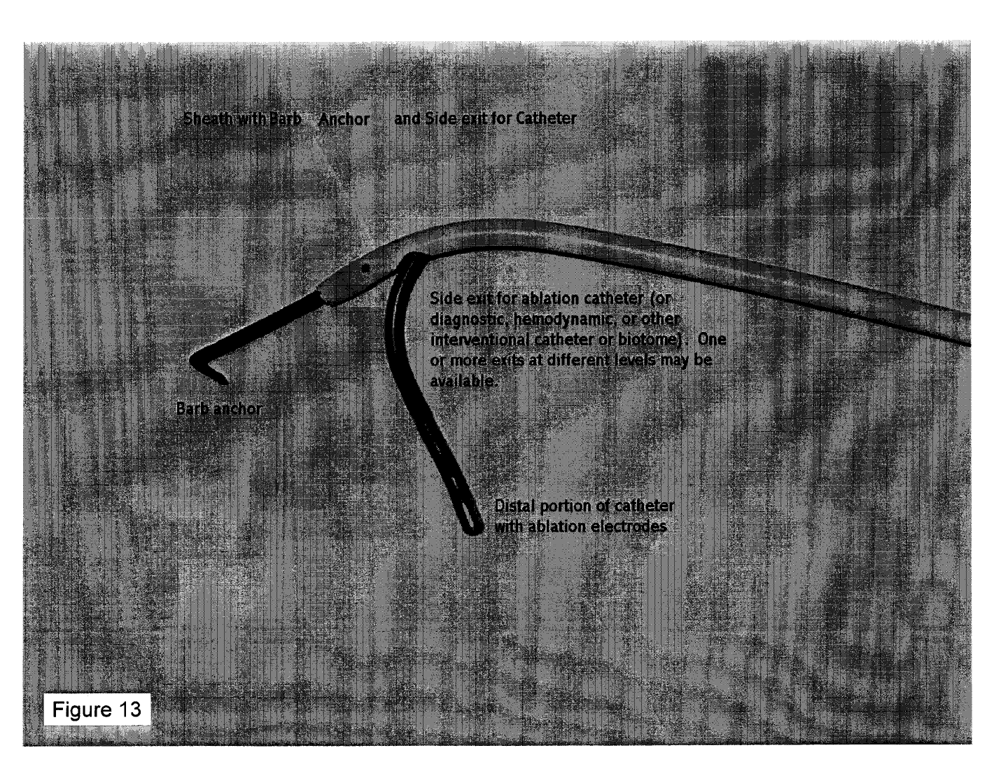

In another embodiment, the anchor is a barb that hooks into the tissue (FIG. 13). Mechanisms for use of the barb include positioning it adjacent to or within the sheath and deploying it, e.g., by moving it laterally and/or axially with respect to the sheath, to hook onto or into an adjacent structure. A barb may also be controlled, so that the backward facing point is deployed or retracted by actuation by the user.

In all embodiments, one or more sensors, e.g., electrodes (see, e.g., U.S. Pat. No. 4,960,134) or pressure or temperature transducers (see, e.g., US 2008/0275367), may be positioned at the distal portion of the anchor. Electrodes allow the measurement of electrograms in order to confirm correct placement of the anchor.

It will also be understood that the size of the components of the anchor employed will depend on the size of the sheath and the tissue to which it attaches. Preferred anchors are sized to attached to tissue in the interior of the human heart.

Rotation Locking Mechanism

The invention also provides locks for arresting the axial and/or longitudinal movement of a catheter relative to the sheath.

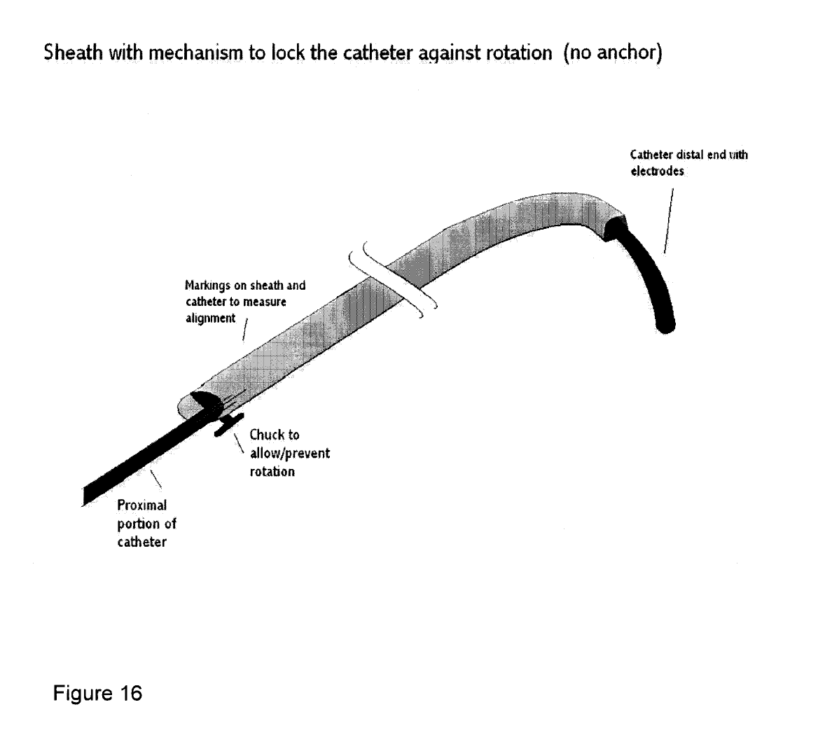

In one embodiment, the proximal end of the sheath features a chuck mechanism, controlled by the physician, which can exert radial pressure on the catheter to lock it in place. This mechanism will prevent both rotational and longitudinal movement as shown in FIGS. 16 and 18-1.

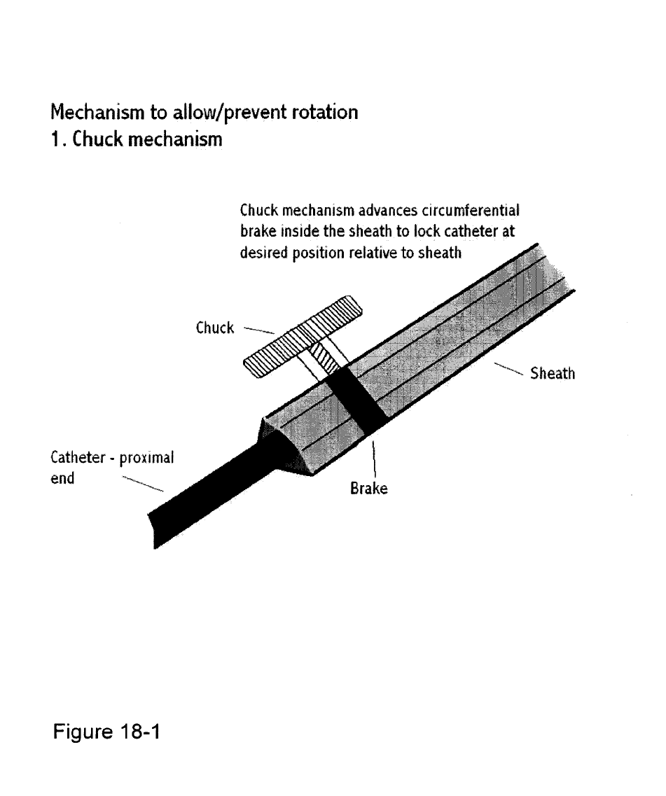

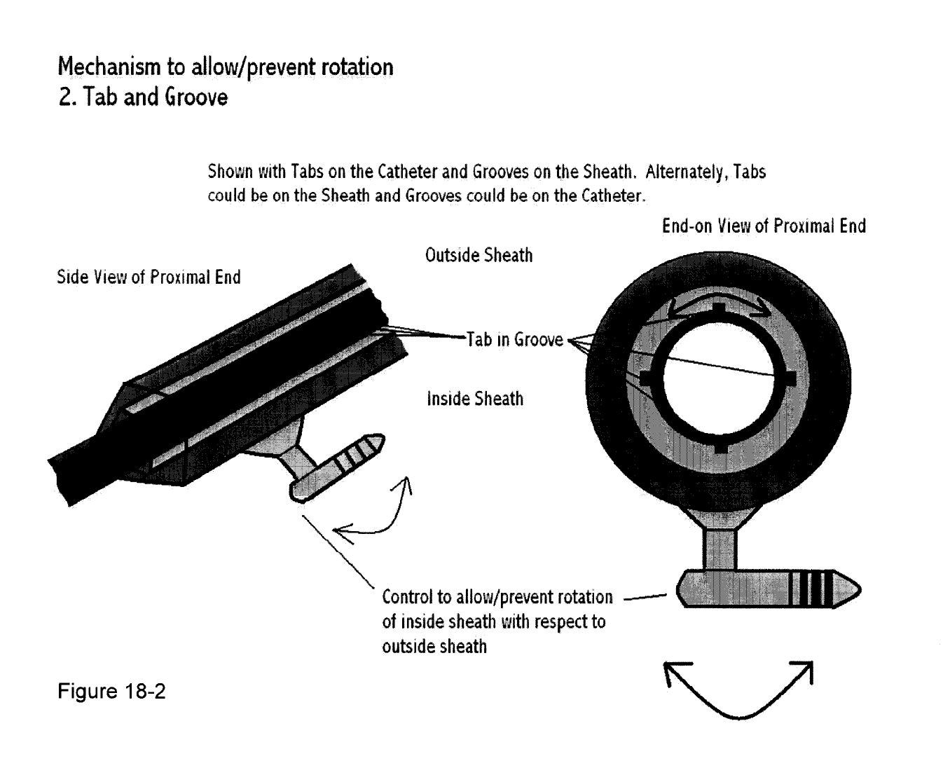

In a second embodiment, the sheath and catheter include an interlocking set of tabs and grooves to control axial movement. For example, the proximal end of the sheath features an inside and an outside section as shown in FIG. 18-2. The inside section is molded with a tab or a groove that mates with a corresponding groove or tab running down the shaft of the catheter. A mechanism on the sheath allows the physician to rotate the inside sheath with respect to the outside sheath, which in turn rotates the catheter. This mechanism can be repeatedly locked or opened by the physician to prevent or allow catheter rotation. In this embodiment, the catheter is prevented from rotation but longitudinal motion is not restricted.

In a third embodiment, the proximal end of the sheath contains a mechanism that is designed with a high degree of static friction, but once that static friction is overcome, the mechanism exerts a low degree of kinetic friction. In this way, the physician exerts sufficient force to overcome the static friction but is then free to rotate the catheter and to move the catheter longitudinally. After the physician has finished moving the catheter, the static friction of the mechanism then prevents the catheter from rotating further. This mechanism may include a spring-loaded stopping plate that gets moved out of the way with enough pressure and then is held out of the way by a detent in the plastic of the sheath, such detent yielding after a period of time and allowing the stopping plate to move back in place.

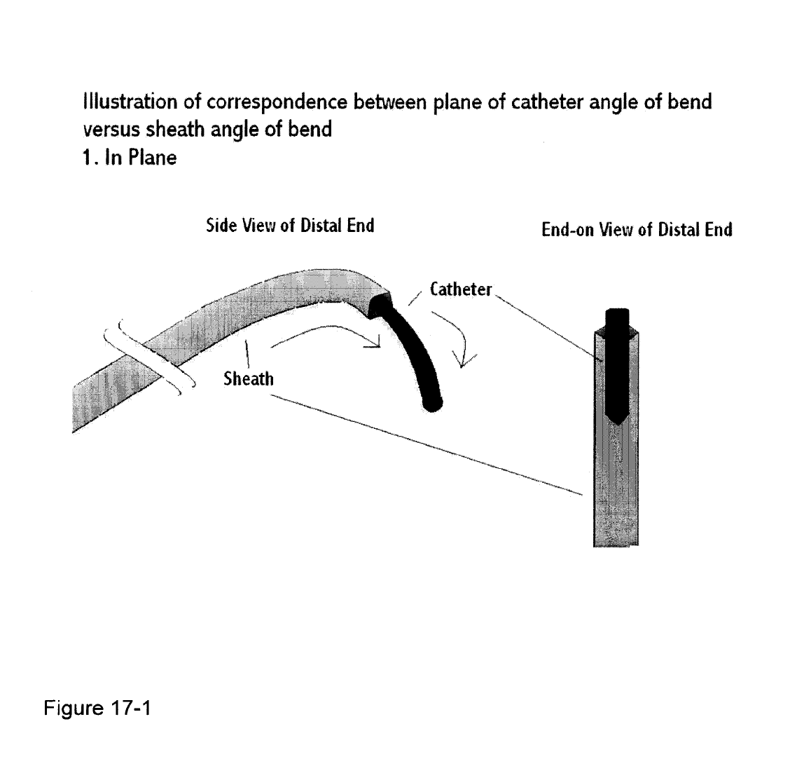

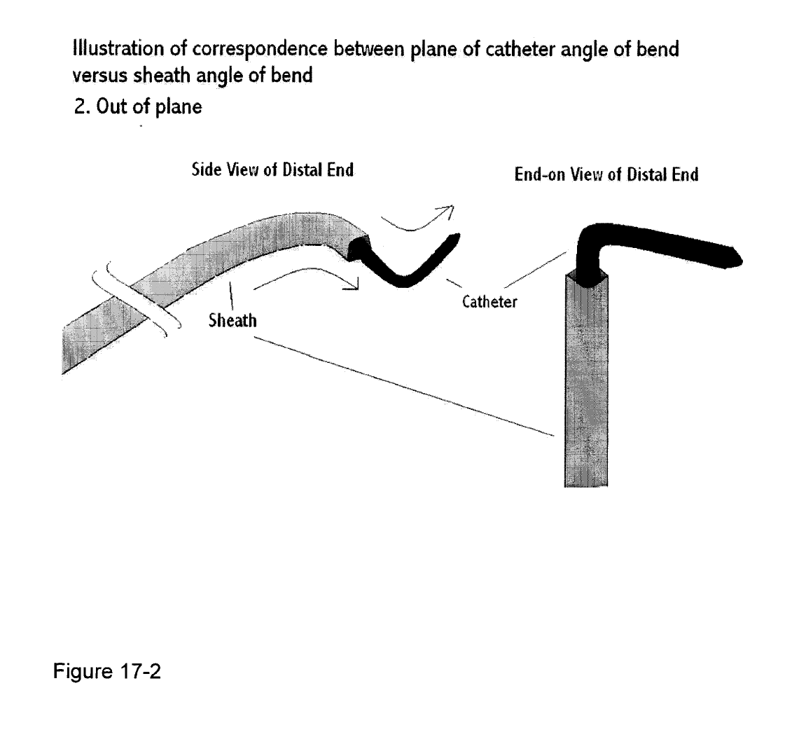

In all of these embodiments, fiducial marks may be molded into (or printed on) the sheath, e.g., that corresponds to line(s) running longitudinally down the shaft of the catheter (FIGS. 14-15). The physician can assess the amount of rotation of the catheter and/or the relative longitudinal movement by looking at the displacement between the reference line on the sheath and the line(s) on the catheter. Knowing the relative axial position of the catheter relative to the sheath allows the user to correctly position the distal end of the catheter, e.g., in plane or out of plane with respect to the curve of the sheath (FIGS. 17-1 and 17-2).

Methods

The sheaths described herein may be inserted into any appropriate lumen. Exemplary lumens include intravascular spaces and spaces within organs (e.g., the heart, lungs and/or bronchi, stomach, rectum, and urinary bladder). The intended use of the sheath will be used to determine the overall dimensions, the number and position of exits for catheters, and the materials employ in its manufacture, all of which are well known in the art. Typically, a sheath may accommodate catheters and other instruments having diameters between 3 and 34 French, e.g., 4-16 French. A preferred catheter diameter is about 4 mm, with a corresponding lumen diameter of about 5-6 mm. The overall length of the sheath is typically between 10 and 100 cm. In a preferred embodiment, the sheath is sized for percutaneous access to the interior of a human heart or sized for access to the epicardium via an introducer of 10 gauge or smaller diameter.

For a given indication, an appropriate catheter will be selected for insertion into a sheath. Examples of catheters include interventional catheters and diagnostic catheters. Exemplary interventional catheters include those for cardiac uses, e.g., ablation, angiography, angioplasty (with or without stenting), permanent pacing, defibrillation leads, and atherectomy. Catheters may also be employed to place permanent sensors with implanted devices for monitoring a physiological function (like cardiac pressure). Catheters for percutaneous intervention may also be employed, e.g., for cardiac, pulmonary, and urinary indications. Ablation catheters are known in the art. Diagnostic catheters include ultrasound probes, Doppler probes, and radiopaque catheters. Diagnostic catheters may also allow indirect or direct visualization of an area, e.g., via x-ray or fluoroscopy, fiber optics, or video camera. Diagnostic catheters may be used to measure blood pressure, blood flow, and electrocardiograms.

A catheter may also be employed to deliver or remove a fluid or other material (e.g., biopsy sample) from a biological lumen. Fluid delivery includes delivery of drugs, pressurizing fluid (e.g., for lung insufflation), and diagnostic agents. Fluid may be removed to reduce local pressure or assay for content, e.g., blood gases. Other types of catheters usable with the invention include central venous catheters, hemodialysis catheters, and urinary catheters (e.g., Foley catheters).

Other interventional catheters or biotomes may be employed with the sheaths of the invention.

### Other Embodiments

All publications, patents, and patent applications mentioned in the above specification are hereby incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. An intraluminal sheath comprising (i) a proximal end, a distal end, and a lumen sized to allow passage of a catheter and extending from the proximal end to the distal end and (ii) an active anchor at the distal end capable of reversibly adhering the sheath to a tissue to provide substantially constant relative position of the sheath to the tissue, wherein said active anchor is selected from the group consisting of a reversibly inflatable balloon comprising a sensor, a deflectable tip, a suction cup, a screw, and a barb.

2. The sheath of claim 1, further comprising a side exit for said catheter at said distal end.

3. The sheath of claim 1, wherein said anchor comprises a sensor.

4. The sheath of claim 3, wherein said sensor is an electrode or pressure or temperature sensor.

5. The sheath of claim 1, wherein said distal end is fixed curved or variably curved.

6. The sheath of claim 1, wherein said sheath further comprises fiducial marks to indicate the axial position of said catheter relative to said sheath.

7. The sheath of claim 1, wherein said anchor is said reversibly inflatable balloon, and said sheath comprises a second lumen for inflating and deflating said balloon.

8. The sheath of claim 1, wherein said anchor is said deflectable tip, and said sheath comprises a control to operate said deflectable tip.

9. The sheath of claim 8, wherein said tip further comprises a barb or screw.

10. The sheath of claim 1, wherein said anchor is said suction cup, and said sheath comprises a second lumen for applying suction to said suction cup.

11. The sheath of claim 1, wherein said anchor is said screw, and said sheath comprises a control to operate said screw.

12. The sheath of claim 1, wherein said anchor is said barb, and said sheath comprises a control to operate said barb.

13. The sheath of claim 1, further comprising a lock to prevent axial and/or longitudinal movement of said catheter relative to said sheath.

14. The sheath of claim 13, wherein said lock comprises a tab or slot that mates with a corresponding tab or slot on said catheter.

15. The sheath of claim 13, wherein said lock comprises a clamp capable of applying radial pressure to said catheter.

16. The sheath of claim 13, wherein said lock has a high degree of static friction between said sheath and said catheter.

17. The sheath of claim 16, further comprising a detent that provides a low degree of kinetic friction once the high degree of static friction has been overcome.

18. An intraluminal sheath comprising (i) a proximal end, a distal end, and a lumen sized to allow passage of a catheter and extending from the proximal end to the distal end and (ii) an active anchor at the distal end capable of reversibly adhering the sheath to a tissue to provide substantially constant relative position of the sheath to the tissue, and (iii) a lock to prevent axial and/or longitudinal movement of said catheter relative to said sheath.

19. The sheath of claim 18, wherein said active anchor is selected from the group consisting of a reversibly inflatable balloon, a deflectable tip, a suction cup, a screw, and a barb.

20. The sheath of claim 18, wherein said lock comprises a tab or slot that mates with a corresponding tab or slot on said catheter.

21. The sheath of claim 18, wherein said lock comprises a clamp capable of applying radial pressure to said catheter.

22. The sheath of claim 18, wherein said lock has a high degree of static friction between said sheath and said catheter.

23. The sheath of claim 22, further comprising a detent that provides a low degree of kinetic friction once the high degree of static friction has been overcome.

24. An intraluminal sheath comprising (i) a proximal end, a distal end, and a lumen sized to allow passage of a catheter and extending from the proximal end to the distal end and (ii) an active anchor at the distal end capable of reversibly adhering the sheath to a tissue to provide substantially constant relative position of the sheath to the tissue, wherein said sheath is sized for percutaneous access to the interior of a human heart.

25. An intraluminal sheath comprising (i) a proximal end, a distal end, and a lumen sized to allow passage of a catheter and extending from the proximal end to the distal end and (ii) an active anchor at the distal end capable of reversibly adhering the sheath to a tissue to provide substantially constant relative position of the sheath to the tissue, wherein said sheath is sized for percutaneous access to a human epicardium via an introducer of 10 gauge or smaller diameter.

26. A method of positioning a catheter in a lumen of a subject, said method comprising:
(a) introducing a sheath of claim 1 into said lumen of said subject;
(b) activating said active anchor to adhere said sheath to a tissue adjacent said lumen of said subject to provide substantially constant relative position of the sheath to the tissue; and
(c) inserting said catheter into said sheath so that said catheter traverses said sheath to said distal end, thereby positioning said catheter.

27. The method of claim 26, wherein said lumen of said subject is within a blood vessel or organ of said subject.

28. The method of claim 26, wherein said lumen of said subject is within the heart of said subject.

29. The method of claim 26, wherein said catheter is an interventional catheter or a diagnostic catheter.

30. The method of claim 26, further comprising delivering a fluid to said lumen or removing a fluid or tissue from said lumen of said subject.

31. The method of claim 26, wherein said anchor is said deflectable tip, and said sheath comprises a mechanical or pneumatic control to operate said deflectable tip.

32. The method of claim 31, wherein said anchor displaces a second tissue away from said catheter.
